(19) United States
(12) Patent Application Publication
(10) Pub. No.: US 2021/0280303 A1
(43) Pub. Date: Sep. 9, 2021

(54) HEALTH CARE FACILITY MONITORING SYSTEM

(71) Applicant: EdjAnalytics LLC, Louisville, KY (US)

(72) Inventors: Susan Olson, Louisville, KY (US); Sean O'Leary, Louisville, KY (US); Casey Ramage, Louisville, KY (US)

(21) Appl. No.: 17/193,393

(22) Filed: Mar. 5, 2021

Related U.S. Application Data
(60) Provisional application No. 62/986,422, filed on Mar. 6, 2020.

(51) Int. Cl. G16H 40/20; G07C 9/00; G06Q 90/00; G06Q 30/02; G06N 5/04; H04W 4/029

(52) U.S. Cl. CPC G16H 40/20; G07C 9/00; G06Q 90/20; H04W 4/029; G06Q 30/0264; G06N 5/04; G06Q 30/0261

(57) ABSTRACT

A monitoring system that includes at least one virtual boundary and assigns persons or mobile devices crossing into the virtual boundary a identifier. The identifier is then monitored for further crossing into and out of the virtual boundary and a categorization is developed based on the frequency, time passage, and other variables. The categorization will generally associate the identifier as an employee, a patient, or a visitor. In addition, the categorization step further includes sub-categorizing types of patients and employees. A monitoring circuit is provided that that generates recommendations related to organization, scheduling, and facility layout configurations, market share opportunities, staffing, hours of operation, quality benchmarks can be provided.

### CROSS-REFERENCE TO RELATED APPLICATION

This United States Utility Application claims the benefit of and priority to U.S. Provisional Patent Application No. 62/986,422, filed Mar. 6, 2020, and titled “HEALTH CARE FACILITY MONITORING SYSTEM”, the entire disclosure of which is hereby incorporated by reference.

### BACKGROUND OF THE INVENTION

### 1. Field of the Invention

The present invention relates a monitoring system for a health care facility or a plurality of health care facilities. More particularly, the present invention relates to a monitoring system for a health care facility that incorporates at least one location aware technology.

### 2. Related Art

This section provides background information related to the present disclosure which is not necessarily prior art.

The medical industry has benefited greatly from advances in technology. These advances in technology have generally been associated with more accurate and less invasive means of a diagnosis and treatment of medical ailments. However, it is less recognized that advances in technology have also improved the medical industry in other ways, such as organizationally. For example, technological improvements have enhanced standardization controls, medical record keeping, inter-building communications, efficient architectural layouts, and scheduling tools. Enhanced standardization controls and medical record keeping provide a streamlined verification process by flagging potential issues in view of personal and family medical histories. In a similar fashion, enhanced architectural layouts, inter-building communications, and scheduling tools have resulted in shorter wait-times for patients, a smaller and more manageable workspace for staff, and a generally more user-friendly experience for all parties involved in a given health care facility. Despite these advancements, however, there are still lingering issues with intra-facility or multi-facility organization, data gathering, data usage, and record keeping.

For example, there is continued difficultly accurately measuring intra and multi-facility aspects, such as readmissions, follow-on services, timing of various treatments, employee to patient ratios, and numerous other aspects. Accurate measurements of these aspects can be ultimately used to make improvements on staffing, hours of operation, quality benchmarks, and market trends. Accordingly, there is a continuing desire to further develop and enhance intra-facility or multi-facility activity monitoring tools and processes.

### SUMMARY OF THE INVENTION

This section provides a general summary of the disclosure and is not to be interpreted as a complete and comprehensive listing of all of the objects, aspects, features and advantages associated with the present disclosure.

According to one aspect of the disclosure, a monitoring system for at least one healthcare facility is provided. The monitoring system comprises a memory and a processor. The memory includes instructions executable by the processor to: generate or identify at least one virtual boundary around at least a portion of the at least one healthcare facility; generate an identifier of a person or a mobile device during a first entry into the at least one virtual boundary; generate timestamp of the first entry and a timestamp of the first exit of the identifier to determine a first occupation period; generate a category of the identifier based on at least one of the occupation period, a frequency of reentries into the at least one virtual boundary, or a time of the day associated with at least one of the timestamps.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected aspects and are not intended to limit the scope of the present disclosure. The inventive concepts associated with the present disclosure will be more readily understood by reference to the following description in combination with the accompanying drawings wherein:

FIG. 1 is a schematic view of a monitoring system;

FIG. 2 is a schematic view of the monitoring system illustrating several virtual boundaries encircling various predefined areas;

FIG. 3 is a schematic view of a monitoring circuit adapted for use in the monitoring system;

FIG. 4 is a flow diagram generally illustrating a method of operating the monitoring system;

FIG. 5A is a flow diagram generally illustrating another method of operating the monitoring system;

FIG. 5B is a flow diagram generally illustrating a continuation of categorization steps provided in FIG. 4 and FIG. 5A;

FIG. 6 is a diagram illustrating series of timestamped entries and exits of a person or mobile device with respect to a series of virtual boundaries;

FIG. 7 is a continuation of FIG. 6 and provides a subpopulation diagram based on a series of timestamped entries and exits;

FIG. 8 is another example diagram that illustrates a series of timestamped entries and exits of a person or mobile device in accordance with yet another aspect of the present disclosure;

FIG. 9 is yet another example diagram that illustrates a series of timestamped entries and exits of a person or mobile device within two non-overlapping separate virtual boundaries;

FIG. 10 is another example diagram that illustrates a series of timestamped entries and exits of a person or mobile device within two non-overlapping separate virtual boundaries that are associated with different healthcare providers or organizations;

FIG. 11 is yet another example diagram that illustrates a series of timestamped entries and exits of a person or mobile device to determine admission and readmission in various healthcare departments; and

FIG. 12 is a diagram that illustrates a series of timestamped entries and exits of a first category of persons or mobile device and a second category of person or mobile devices within at least one virtual boundary to determine patient visit to employee/nurse ratio.

### DESCRIPTION OF THE ENABLING EMBODIMENT

Example embodiments will now be described more fully with reference to the accompanying drawings. In general, the subject embodiments are directed to a health care facility monitoring system, and a method of operating same. However, the example embodiments are only provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Referring to the Figures, wherein like numerals indicate corresponding parts throughout the views, the health care facility monitoring system is intended for tracking and categorizing of persons entering one or more virtual boundaries in order to make recommendations for providing enhanced intra-facility or multi-facility activity.

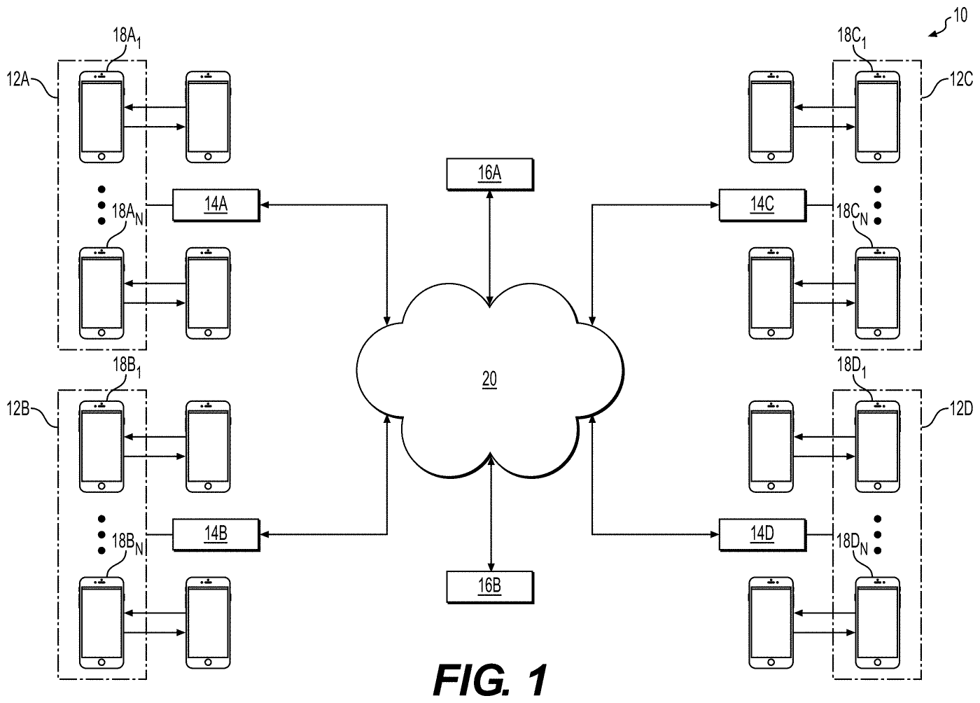

Referring initially to FIG. 1, a schematic view of the monitoring system 10 is provided. The monitoring system 10 includes at least one virtual boundary 12, for example, a geofence boundary, that surrounds an area or region associated with a health care facility. The monitoring system 10 further includes at least one local computing device 14 and/or a remote computing device 16 that is configured to generate a identifier representing a person or mobile device that exits or enters the at least one virtual boundary 12. The identifier may be associated with a particular mobile device 18, such as a mobile phone 18. As a person carries the mobile phone 18 into the virtual boundary 12, one of the local computing device 14 and/or a remote computing device 16 attaches an identity tag to the person/device via a unique property of the mobile device 18, e.g., an IP address. For example, the virtual boundary 12 may be associated with a wireless network range, wherein every time a mobile device 18 enters therein, a wireless network inquiry is made by the mobile device 18 and the IP address or a similar unique property is tagged with an associated entry time and an exit time.

Other aspects of the virtual boundary 12 may include GPS and RFID tracking of the mobile device 18. As will be described in detail below, each identity tag may only function as an identifier (i.e., no additional information from the mobile device 18 or person is collected other than the time of entering and exiting various virtual boundaries 12). Entry and exit data can be extrapolated to determine the frequency (relative or absolute) that the mobile device 18 enters the virtual boundary 12, the length of time the mobile device 18 stays within the virtual boundary 12, and common routines between two or more virtual boundaries 12A, 12B, . . . 12N (N representing all natural numbers). Using the generated log of data, software located at the local computing device 14 and/or the remote computing device 16 can determine the amount of foot traffic through or between various locations in the healthcare facility (defined by virtual boundaries) and can categorize individual persons or mobile devices 18 based on their routines. For example, a person or mobile device 18 that enters the virtual boundary 12 three times in a month may be identified as a visitor while a person who enters the virtual boundary 12 in similarly timed 10-hour increments Monday through Friday may be identified as a worker/employee via these absolute readings. In addition, relative readings may also be used for form categorizations, for example, spikes or lulls in the frequency of crossing a virtual boundary of an individuals may be used to form categories, e.g., sales persons, rotating or visiting staff, patients at certain stages of treatment, etc. In some embodiments, if a person breaks a pattern and enters, exits, and reinters a virtual boundary within a predetermined period, e.g., five minutes, the reading may be nullified or not categorized as absolute when forming a recommendation or further analysis. In some embodiments, a person may regularly leave a virtual boundary to get lunch, in such instances the absolute reading of entries into the virtual boundary may skew the statistical model, thus these readings may also be nullified or not categorized as absolute when forming a recommendation or further analysis. In some embodiments, the monitoring system may have threshold predetermined lengths of time between exit and reentry of the virtual boundary 12 that are not used to categorize the person, mobile device, or for an absolute statistical model.

The monitoring system 10 may utilize the data obtained and provides one or more recommendations via the local computing device 14 and/or the remote computing device 16. As will be described in greater detail below, the recommendations may be directed to organization, scheduling, and facility layout configurations, market share opportunities, staffing, hours of operation, quality benchmarks, or combinations thereof. The local computing device 14 and/or the remote computing device 16 may be in communication with a server 20 that stores the data from each virtual boundary that is present in the system 10 and stores it for later access and extrapolation.

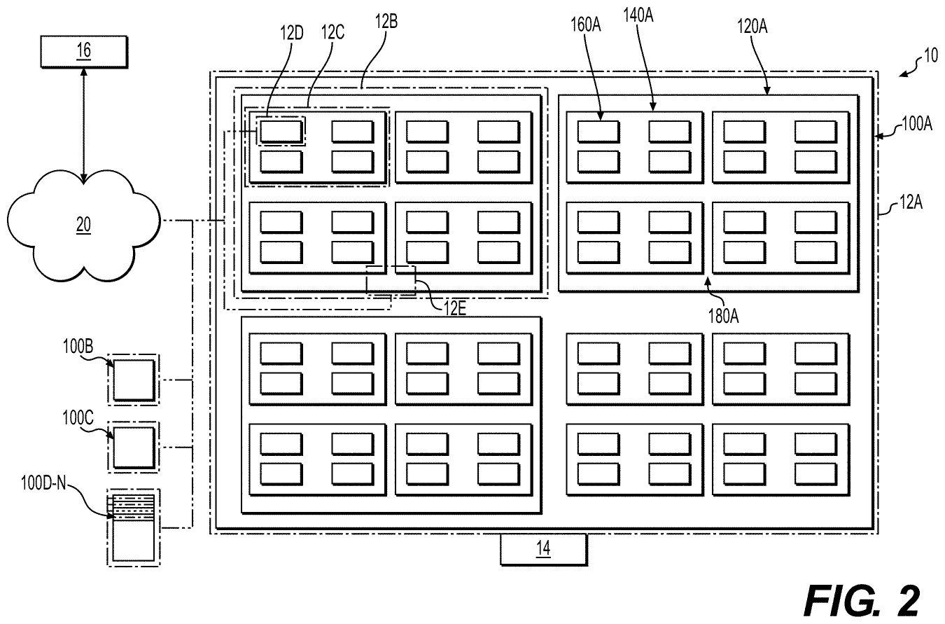

The virtual boundaries 12 may surround or encircle a number of predefined areas and larger virtual boundaries may surround several smaller virtual boundaries. As best illustrated in FIG. 2, a schematic view of several virtual boundaries encircling various predefined areas is illustrated. The virtual boundaries may encircle one or more regions 100, medical campuses 120, medical buildings 140, departments 160, and/or traffic locations 180. Each region 100 represents a geographical area with two or more medical campuses 120, medical buildings 140, departments 160, or combinations thereof which may be operated by the same or different providers. In some embodiments, each medical campus 120 includes at least two medical buildings 140 operated by a singular provider, and each building 140 includes one or more departments 160. The traffic locations 180 represent public or private roadway entrances, crossroads, parking, building entrances, and/or hallways that are associated with the campus 120, medical building 140, department, or combinations thereof. The intra-building virtual boundaries may be spaced vertically by floor, horizontally, or combat ions thereof. The server 20 may gather data on any number of regions 100A through 100N (N representing all natural numbers), any number of medical campuses 120, any number of medical buildings 140, any number of departments 160, any number of traffic locations 180, or a combination thereof (generically referred to as “healthcare facility”). Data may be stored on one or both of the local computing device 14 and the remote computing device 16.

With continued reference to FIG. 2, a large virtual boundary 12A surrounds an entire region 100A, a smaller virtual boundary 12B is enclosed by the large virtual boundary 12A and surrounds a medical campus 120A, a next smaller virtual boundary 12C is enclosed by the virtual boundary 12B and surrounds a medical building 140A, and a next smaller virtual boundary 12D is enclosed by the virtual boundary 12C and surrounds a department 160A. In addition, another virtual boundary 12E is also enclosed by virtual boundary 12B and surrounds a traffic location 180A that is represented as a crossroad. Locations of virtual boundaries 12A through 12E are not limited to the illustrative example and may be located around some or select of the various other areas provided including multiple medical campuses 120, medical buildings 140, departments 160, traffic locations 180, of combinations thereof.

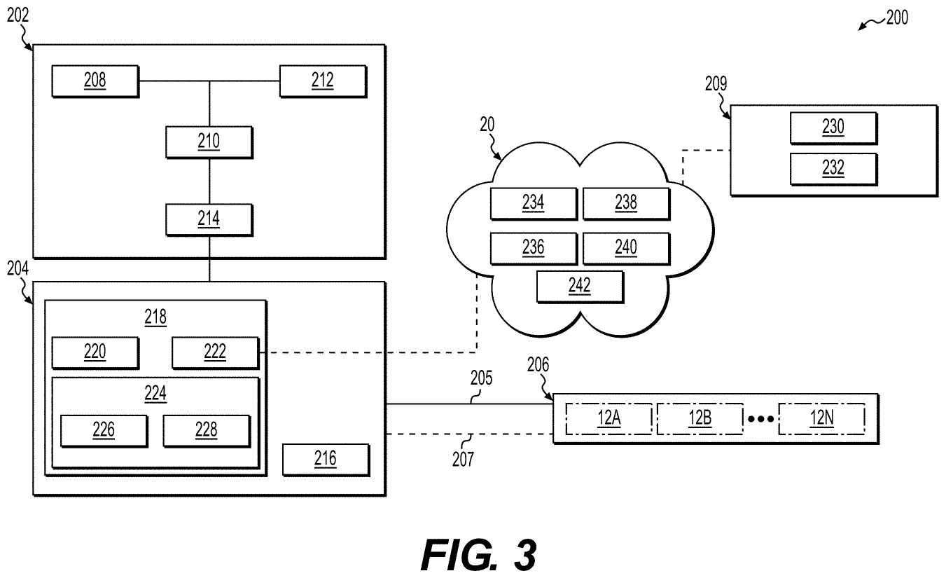

As best illustrated in FIG. 3, the monitoring system 10 includes at least one monitoring circuit 200 located within the local computing device 14, the remote computing device 16, or combinations thereof. The various elements provided therein allow for a specific implementation. Thus, one of ordinary skill in the art of electronics and circuits may substitute various components to achieve a similar functionality. The monitoring circuit 200 includes a power system 202, a GCU circuit 204, a location-aware system 206, and server 20. The location-aware system 206 may incorporate one or more geofences or other location-aware technology boundaries. The power system 202 includes a power supply circuit 208 that is monitored via a power supervision circuit 210 and a back-up power circuit 212 (associated with a back-up battery or generator) that may be primarily charged via the power supply circuit 208. A power testing unit 214 tests for current from the power supply circuit 208 to ensure that power is being transmitted to the GCU circuit 204. In the event of a power failure, the power-testing unit 214 may utilize the back-up battery or generator for initiating various protocols in the GCU circuit 204, such as continued operation of the monitoring circuit 200. Moreover, in power failure events wherein the location-aware systems can no longer be effectively monitored, a visual alarm unit 216 is located on the GCU circuit 204 such that it can generate a warning such as power failure via a user interface on the local computing device 14 and/or the remote computing device 16. Operation of the power system 202 is via a controller 218 located in the GCU circuit 204.

The controller 218 includes a processor 220, a communications unit 222 (for example associated with wired or wireless internet connection), and a memory 224 having machine-readable non-transitory storage. Programs and/or software 226 are saved on the memory 224 and so is data 228 obtained via the many virtual boundaries 12A through 12N and/or the server 20. The memory 224 may comprise a single disk or a plurality of disks (e.g., hard drives), and includes a storage management module that manages one or more partitions within the memory 224. In some embodiments, memory 224 may include flash memory, semiconductor (solid state) memory or the like. The memory 224 may include Random Access Memory (RAM), a Read-Only Memory (ROM), or a combination thereof. The memory 224 may include instructions that, when executed by the processor 220, cause the processor 220 to, at least, perform the systems and methods described herein. The processor 220 carries out instructions based on the software 226 and data 228, for example, providing a recommendation based on at least one of enhanced internal and external organization, scheduling, and facility layout configuration. Additionally, or alternatively, the controller 218 may include any suitable number of processors, in addition to or other than the processor 220. Communications between the GCU circuit 202 and the server 20 are carried by the communications unit 222, allowing both transmittal and receipt of information. As such, software 226 and data 228 may be updated via instructions from the server 20. In one example embodiment, the server 20 is further connected to a remote computing device circuit 209 (associated with the remote computing device 16) for initiating software updates 230 and transmitting other assigned data 232 to local computing devices 14. The location-aware system 206 is connected to GCU circuit 204 with a wireless connection 205 and/or a wired connection 207. The location-aware system 206 can include at least one location-aware technology such as a geofence and may include a plurality of geofences 12A through 12N or other location-aware technologies. Data retrieved by the location-aware system 206 may be stored locally in memory 224 or remotely. As discussed previously, individual virtual boundaries 12A through 12N may operate via any type of location-aware technology including but not limited to wireless inquiries, GPS, RFID, geofencing, and other methodologies.

With continued reference to FIG. 3, the server 20 may be a storage server that that stores data from more than one location-aware system 206 via one or more GCU circuit 204. In some embodiments, data stored in the server 20 may be categorized as proximity data 234, operations schedule data 236, passage of time data 238, meta data 240, and historical data 242. Data stored in server 20 may also be stored in memory 224, which may be transmitted and/or otherwise directly generated as persons or mobile devices 18 cross the at least one virtual boundary 12. The proximity data 234 may include the distance between two or more non-overlapping or overlapping virtual boundaries 12. The operations schedule data 236 may be related to shift changes in staff, delivery periods, hours of operation of various departments, non-medical shop and cafeteria hours, or combinations thereof. The operations schedule data 236 may further be related to facility layout and treatments associated with certain areas with the medical facility. The passage of time data 238 may include how long persons or mobile devices 18 remain located within the respective virtual boundaries 12. The meta data 240 may include instances of persons or mobile devices 18 that enter two or more virtual boundaries that are overlapped or non-overlapped, the number of people or mobile devices 18 entering into a virtual boundary 12 in a time period, trends of at least one person or mobile device 18 moving between two or more virtual boundaries, or combinations thereof. The historical data 242 may include previous categorizations, identifiers, frequency of entries, timestamps, and occupation periods of persons or mobile devices 18, e.g., employees, patients, caregivers, facility service contractors, and visitors. The transfer of data between the GCU circuit 204, the server 20, and the computing device circuit 209 is preferably real-time or near real-time. As such, historical data 242 may be changed as designations of persons or mobile devices 18 change from repeated contact with one or more virtual boundaries.

The schematic diagram of the circuit 200 in FIG. 3 is provided as just one example, it should be appreciated that the various components can be located locally in the local computing device 14, remotely in the remote computing device 16, or combinations thereof without departure from the scope of the subject disclosure. The local computing device 14 and/or the remote computing device 16 may include a user interface such as, without limitation, a monitor and a keyboard, a touchscreen, a mobile device, or combinations thereof.

Accordingly, systems and methods, such as those described herein, configured to provide monitoring and recommendations of at least one health care facility, may be desirable.

In some embodiments, the systems and methods described herein may be configured to generate or identify at least one virtual boundary 12 around at least a portion of the at least one healthcare facility in accordance with a step 252 of method 250. For example, the processor 220 and/or one of the computing devices may generate or identify at least one virtual boundary 12 that is otherwise generated around at least a portion of the at least one healthcare facility (e.g., a region, a medical campus, a medical building, a department, a traffic location, or combinations thereof). The at least one virtual boundary may encircle one or more regions 100, medical campuses 120, medical buildings 140, departments 160, traffic locations 180, or combinations thereof.

In some embodiments, the systems and methods described herein may be configured at 254 to generate an identifier of a person or a mobile 18 device during a first entry into the at least one virtual boundary 12. For example, one of the local computing device 14 and/or a remote computing device 16 attaches an identity tag or identifier to the person or mobile device via a unique property of the mobile device 18, e.g., an IP address. For example, the virtual boundary 12 may be associated with a wireless network range, wherein every time a mobile device 18 enters therein, a wireless network inquiry is made by the mobile device 18 and the IP address or a similar unique properties.

In some embodiments, the systems and methods described herein may be configured at 256 to generate timestamp of the first entry and a timestamp of the first exit of the identifier to determine a first occupation period. For example, the processor 220 and/or one of the computing devices may log the time of day that the person or mobile device 18 enters and exits the at least one virtual boundary 12. A period of time between entry and exit into the at least one virtual boundary 12 may be equal to the occupation period.

In some embodiments, the systems and methods described herein may be configured at 258 to generate a category of the identifier based on at least one of the occupation period, a frequency of reentries into the at least one virtual boundary, or a time of the day associated with at least one of the timestamps. For example, the processor 220 and/or one of the computing devices may generate a category based on the occupation period wherein a person or mobile device 18 that enters the virtual boundary 12 three times in a month may be identified as a visitor or patient while a person who enters the virtual boundary 12 in similarly timed 10-hour increments (or 4, 6, 8, or 12-hour increments) Monday through Friday (or other patterns of days of the week) may be identified as a worker/employee. Similarly, a person or mobile device 18 that enters the virtual boundary 12 for an hour (or a shorter period of time than 4 hours, 3 hours, 2 hours, etc.) on regular intervals may be categorized as a service person and a person or mobile device 18 that enters the virtual boundary 12 for differing time periods on irregular intervals may be categorized as a visitor. As another example, a person or mobile device 18 that enters the virtual boundary 12 for more than 12 hours (or 14, 16, 18, or more hours) or for irregular times within a service period associated with the at least one virtual boundary may be categorized as a patient. As another example, a person or mobile device 18 that enters the virtual boundary 12 at a time associated with changing shifts may be associated with an employee. The categorization may further implement data from server as described herein.

In some embodiments, the systems and methods described herein may be configured at 260 to generate a category of the identifier that includes at least one of a patient, a visitor, a service person, and an/or employee, which may include treatment providers (e.g. doctors, nurses, techs), including the types of treatment associated with a virtual boundary, and non-treatment providers (e.g., cafeteria staff, shop staff, and hospital administration). For example, the processor 220 and/or one of the computing devices may categorize the person or mobile device 18 based on occupation periods, timestamps, and frequencies as described above. In some embodiments, the frequency of reentries into the at least one virtual boundary 12 includes additional occupation periods.

In some embodiments, the systems and methods described herein may be configured at 262 to save in the memory the timestamps and occupation periods associated with the identifier and generate changes in the category of the identifier associated with the continuing timestamps and occupation periods after then first entry and the first exit. For example, after generating an initial category, repeated timestamps and occupation periods may be compared with the above categorization techniques, wherein trends may develop that are different than an initial categorization.

In some embodiments, the systems and methods described herein may be configured at 264 to generate a first predetermined threshold of time wherein if an occupation period or a period of time between occupation periods is less than the first predetermined threshold it is not used for the purpose of categorization. Such steps may be via the processor 220 and/or one of the computing devices.

In some embodiments, the systems and methods described herein may be configured at 266 to generate a status of the identifier if the occupation period or the period of time between occupation periods is less than the first predetermined threshold, the status of the identifier may include at least one of the identifier being lost in the at least one medical facility, the identifier leaving for food, the identifier looking for parking, or the identifier entering and traversing the at least one virtual boundary to enter an area outside of the at least one virtual boundary. For example, if the identifier (a person or mobile device 18) enters the virtual boundary 12 for less than an hour, it may be determined that the identifier is not any of the categories associated with the virtual boundary 12. Similarly, if an identifier leaves the virtual boundary 12 and returns within a short time period (1 hour or less, 2 hours or less), it may be determined that the identifier is leaving the virtual boundary 12 for food or other purposes that do not directly relate to categorization without further trends. Such steps may be via the processor 220 and/or one of the computing devices.

In some embodiments, the systems and methods described herein may be configured at 268 to generate a plurality of virtual boundaries (12A-12N) and generate a link between the plurality of virtual boundaries (12A-12N) every time an identifier travels between two or more of the virtual boundaries. For example, the processor 220 and/or one of the computing devices may generate or otherwise identify a plurality of virtual boundaries (12A-12N) an generate a link between the virtual boundaries (12A-12N) every time an identifier travels between two or more of the virtual boundaries. These links may be saved in memory and trends can be generated to locate trends in movement between the virtual boundaries (12A-12N).

In some embodiments, the systems and methods described herein may be configured at 270 to generate a recommendation based on the link between at least two virtual boundaries (12A-12N) for at least one of facility layout or the placement of signs to guide traversal between the at least two virtual boundaries (12A-12N). For example, the processor 220 and/or one of the computing devices may recommend a more efficient facility layout that may be implemented in the at least one medical facility associated with the virtual boundaries (12A-12N) or future constructions of additional medical facilities. Similarly, a recommendation for the placement of signs may be generated to include directional information for common travel paths between the virtual boundaries (12A-12N).

In some embodiments, the systems and methods described herein may be configured to generate a recommendation for the placement of at least one sign that includes a recommendation in or near a first virtual boundary 12A of the at least two virtual boundaries (12A-12N) related to services rendered in a second virtual boundary 12N of the at least two virtual boundaries (12A-12N) and the recommendation includes signs for one or more identifiers traveling to the second virtual boundary 12N to avoid the first virtual boundary 12A. For example, the processor 220 and/or one of the computing devices may recommend identifiers traveling to a virtual boundary associated with a contagious medical condition to avoid other areas of the at least one medical facility to minimize risk of spread. In some embodiments, the sign is generated digitally on a screen in the medical facility or on the mobile device associated with the identifier.

In some embodiments, the systems and methods described herein may be configured at 272 to generates an advertisement in a first virtual boundary 12A of the at least two virtual boundaries (12A-12N) related to services rendered in a second virtual boundary 12N of the at least two virtual boundaries (12A-12N). For example, the processor 220 and/or one of the computing devices may generate an advertisement related to healthy lifestyles associated with the medical conditions associated with the second virtual boundary 12N. In some embodiments, the advertisement is generated digitally in response to a time of day associated with busy travel between the at least two virtual boundaries (for one or more identifiers) or a time stamp of entry of the identifier into one of the plurality of virtual boundaries. The advertisement may be on a screen in the at least one medical facility or the mobile device 18 associated with the identifier.

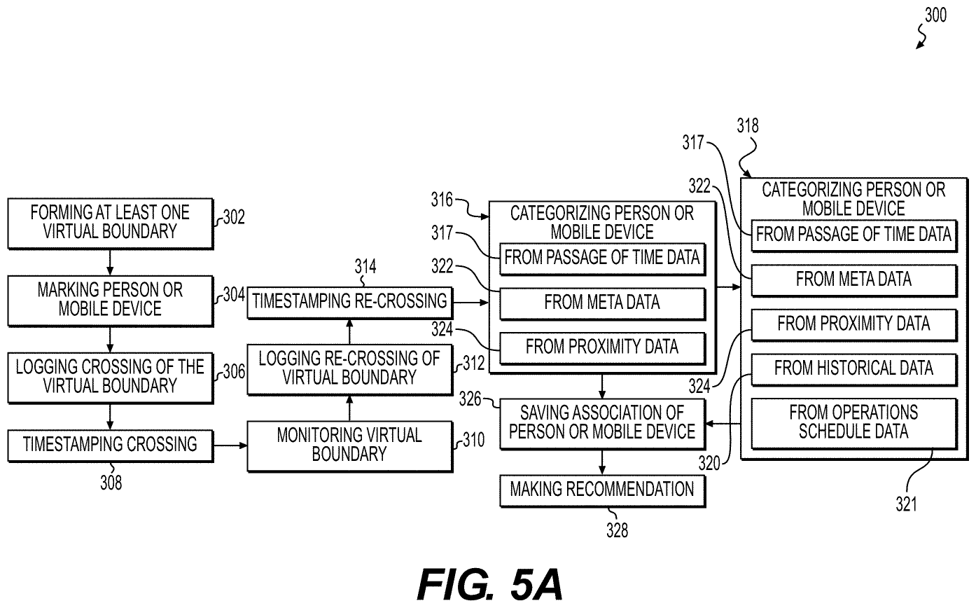

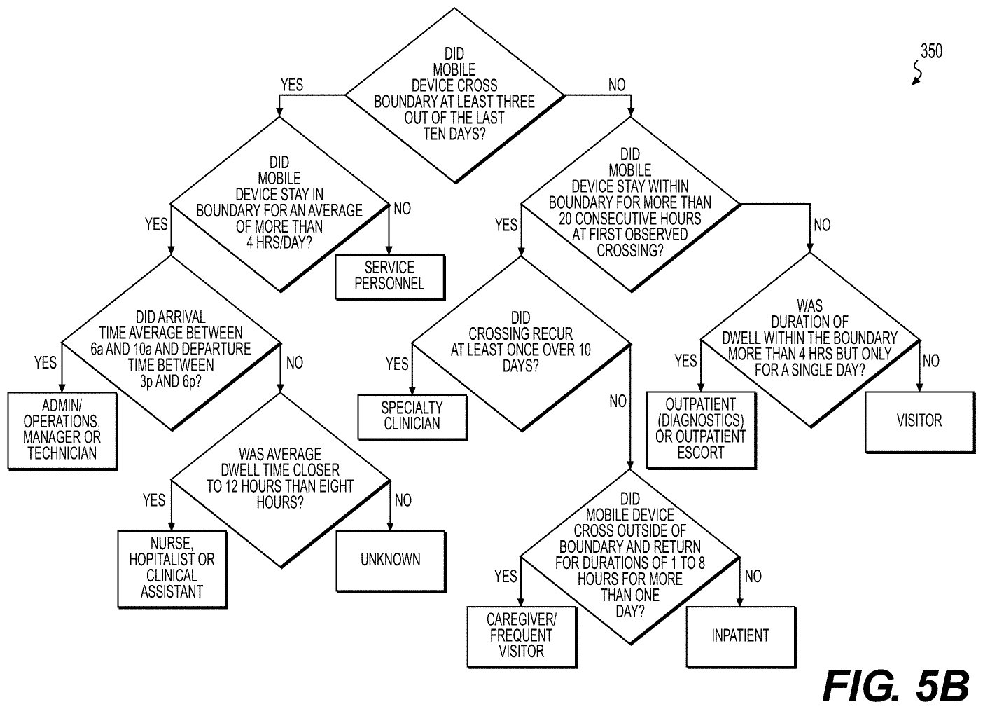

In some embodiments, the systems and methods described herein may be configured at 274 to generate a plurality of additional identifiers of additional persons or a mobile devices (18A-18N) and further generates a category of each of the identifiers based on at least one of the occupation period, a frequency of reentries into the virtual boundary, or a time of the day associated with at least one of the timestamps. Such steps may be via the processor 220 and/or one of the computing devices. In some embodiments, steps of 274 may include steps 258 through 266. In some embodiments, at least steps 258 through 266 and 274 may include and/or be interchanged with steps provided in FIG. 5B. In some embodiments, at least steps 258 through 266 and 274 may incorporate data from the server.

In some embodiments, the systems and methods described herein may be configured at 276 to generate a quotient of at least two categories of identifiers and further generate a recommendation for a target quotient of the at least two categories of identifiers. For example, the processor 220 and/or one of the computing devices may compare the number of identifiers categorized as patients and the number of identifiers categorized as employees (e.g., care providers). In some embodiments, a notification is generated if a quotient between the at least two categories of identifiers is outside of a predetermined threshold. For example, a notification may be generated if a region in the virtual boundary 12 is understaffed beyond a standardized threshold. Standardized thresholds described herein may be provided by a third-party and may further include medical industry requirements.

In some embodiments, the systems and methods described herein may be configured at 278 to generate a notification of readmission when a predetermined length of time passes between occupation periods. For example, the processor 220 and/or one of the computing devices may associate a medical conditions with the at least one virtual boundary and a standardized time of services typically required to treat the associated medical condition. If a predetermined length of time passes similar to the standardize time of services and there is a period absence of the identifier in the at least one virtual boundary 12 the identifier may be designated treated and a later reentry or series of reentries associate with continuing treatment may be designated as readmission. In some embodiments, a recommendation for a target limit of readmissions is generated. For example, the recommendation for target readmissions may include a standardized amount of readmissions for a particular type of treatment that is compared to the identified number of readmissions of the identifier (or a plurality of identifiers). Departments associated with the at least one virtual boundary may then be reviewed if the number of readmissions is above or beyond a threshold from standard.

In some embodiments, the systems and methods described herein may be configured at 280 to encrypt the identifier so that the identifier does not include any personal information that could be used to ascertain a personal identity. For example, the processor 220 and/or one of the computing devices may encrypt an IP address or other feature associated with the personal or mobile device 18 such that the identifier does not contain personal information beyond those necessary to perform the systems and methods described herein.

In some embodiments, the systems and methods described herein may be configured at 282 to generate the at least one virtual boundary 12 and the at least one virtual boundary 12 includes a first virtual boundary 12A around at least a portion of a first healthcare facility and a second virtual boundary 12N around at least a portion of a second healthcare facility and generate a plurality of additional identifiers of additional persons or a mobile devices (18A-18N) and further generate a category of each of the identifiers based on at least one of the occupation period, a frequency of reentries into the virtual boundary, or a time of the day associated with at least one of the timestamps. For example, the processor 220 and/or one of the computing devices may generate a category for each person or mobile device (18A-18N) based on the methods described herein. In some embodiments the first health care facility may be owned by a different provider than the second health care facility.

In some embodiments, the systems and methods described herein may be configured at 284 to generate a quotient between the number of identifiers associated with the first medical facility and the number of identifiers associated with the second medical facility. For example, the processor 220 and/or one of the computing devices may generate data related to the numbers of identifiers and the numbers of each category. Such information may be used to generate market-share between at least two medical facilities and provide recommendations to obtain greater market share based on differences between the at least two medical facilities. In some embodiments, the recommendation may include recommending to at least one of the at least two medical facilities or at least one of the mobile devices (18A-18N) of availability for another medical facility in various situations, such as emergency situations wherein medical service availability is strained and/or situations to consolidate contagious conditions at one or more medical facilities.

In some embodiments, the system 10 may include a method 300 for categorizing personnel or mobile devices 18 as illustrated in FIG. 5A. The method 300 may be carried out by at least one of the processor 220 and one of the computing devices. At 302 the method 300 begins by forming at least one virtual boundary around at least one of a region, a medical campus, a medical building, a department, and a traffic location. At 304, the method 300 continues by marking a person or a mobile device crossing or entering one of the virtual boundaries with a non-personalized identifier (such as a number or other encrypted code via software 226) and logging 306 the crossing, for example, into memory. At 308, the crossing log entry is timestamped. At 310, the method 300 may continue by monitoring the crossed virtual boundary until the person or a mobile device re-crosses the virtual boundary wherein the re-crossing or exit is logged at 312. The step 312 of logging the re-crossing event is followed by an additional timestamp at 314. At 316, the method 300 continues by categorizing the person or mobile device. For example, the method may include categorizing via passage of time data at 317.

In some embodiments, if the timing between entry and exit is over, under, or within a first threshold (e.g., under 4 hours or over 12 hours) the person or mobile device (e.g., identification number) is categorized as a patient or visitor. In some embodiments, if the timing is over, under, or within a second threshold (e.g., timing between entry and exit is over 24 hours), the person or mobile device is categorized as a patient. In some embodiments, if the timing is over, under, or within a third threshold (e.g., entry and exit is between 4 and 12 hours), then the person or mobile device is categorized as a patient, visitor, or employee. In some embodiments, if the timing is under a fourth threshold (e.g., under 10 minutes), the person or mobile device is categorized as a visitor or person that is lost.

After the initial categorization at 316, a second categorization step takes place at 318 when the person or mobile device crosses the virtual boundary at least one additional time. In some embodiments, steps 316 and 318 may also account for which portions of the virtual boundary the person or mobile device is entering or exiting, e.g., if a person or mobile device crosses a portion of the virtual boundary (such as a geofence) next to a visitor or employee parking. This may be accomplished by having additional virtual boundaries around respective parking lots. Next, the categorization 316 and the categorization 318 are compared and if there are discrepancies, then a step 320 of historical data review takes place wherein the mean and/or median timing between entry and exit may be compared to the afore-described thresholds. The historical data review at 320 may include comparing a series of entry timestamps of the associated person or device and/or comparing a series of entry timestamps, exit timestamps, occupation periods, frequencies of entry or combinations thereof. At 321, the method may further include comparing the operations schedule data with the timestamps of entering and exiting the virtual boundary (or occupation periods with the virtual boundary) and the services associated with the virtual boundary. Thus if an identifier has a schedule similar to shifts associated with a virtual boundary they may be categorized as an employee and may be further categorized as a type of employee.

In some embodiments, at 322, the method 300 may include a meta data review, in one or both steps 316 and 318, which compares the entry and exit timestamps of one person or mobile device with those of other persons or mobile devices, wherein the entry and exit during a high traffic period may be indicative of a work schedule change. In some embodiments, the method 300 may further include a proximity data review at 324, in one or both steps 316 and 318, that tracks the entry and exit of a person or mobile device through at least two virtual boundaries, wherein a similar route or a reoccurring transition between routes is indicative of a work schedule in multiple regions, campuses, buildings, and departments. If the passage of time data is short (e.g., less than an hour) for each transition between locations, it may be indicative of a sales or delivery personal. However, if the passage of time data is longer (e.g., over an hour but less than four hours) for each transition it may be further indicative of a janitorial staff. In some embodiments, if the passage of time data is longer yet (e.g., over four hours) for each transition it may be indicative of a medical staff rotation (e.g., a nurse, a doctor, or a tech).

Once a general categorization is reached, the method 300 continues by associating the person or mobile device with a department or service at 326. For example, if the virtual boundary surrounds a cancer treatment department, orthopedic department, or cardiovascular department reentries or the occupation period may be used to associate the person or mobile device with those medical services. Next, a recommendation at 328 may be generated based on one or both the absolute or relative readings. For example, the relative crossing frequency readings of one particular virtual boundary may temporarily spike during certain times of the day, different days, or different times of the year. In addition, certain relative readings can be identified as not being used in the “absolute readings” as previous explained. Relative readings may also be used assist in identifying stages of treatment and readmissions. Similarly, the absolute crossing frequency readings may show certain locations/facilities being under or over utilized when compared to similar location/facilities.

The recommendation at 308 may include designating an area overstaffed or understaffed; providing additional wheel chairs, shops, or other services in locations with heavy traffic associated with patients or visitors; placing maps or directional signs in departments with common categorizations of lost visitors or patients; placing signs related to healthy lifestyle choices, psychological therapists, etc., in associated high traffic travel paths or areas with at risk patients; moving departments closer to one another in current or future facilities. The recommendation step 328 may further include generating a data notification related to employee turnover or patient readmission via meta data between regions, campuses, buildings and also providing a recommendation for improvements to efficiency based on this information. In addition, the recommendation step 328 may further note that certain patients in one department provided by a first provider have longer stays than patients in a different department in the same field by the first or a second provider. Recommendation step 328 may further yet include categorizing readmissions and comparing the number of readmissions to inter and intra facility statistics for forming inter and intra quality benchmark recommendations. The categorization step 318 may repeat indefinitely so long as the person or mobile device continues to cross the virtual boundary.

In some embodiments, the recommendation 328 may further include providing details about market share opportunities in a geographic region, occurrences of patients using more than one health provider, absolute and relative market share maintained by an organization, the rate of hospital readmissions to medical campus, building, or department owned by another organizational entity. In some embodiments, the recommendation 328 may further include the length of time during stays from an original admission and follow-up visits, diagnostic information associated with the original treatment and readmission, statistics and analytics about times of day and/or days of week a patient visits medical campus, building, or department.

In some embodiments, the recommendation 328 may further include generating and relaying differences in times of day and/or days of week across organizational ownership and geographical region, visits to facilities inside against those outside of an organizational ownership for follow-up visits after a hospitalization, differences in length of stay among hospitals owned by different organizations, differences in patient time required for outpatient procedures or office visits at different facilities, differences in employee to patient ratios, staffing patterns, and other employment information.

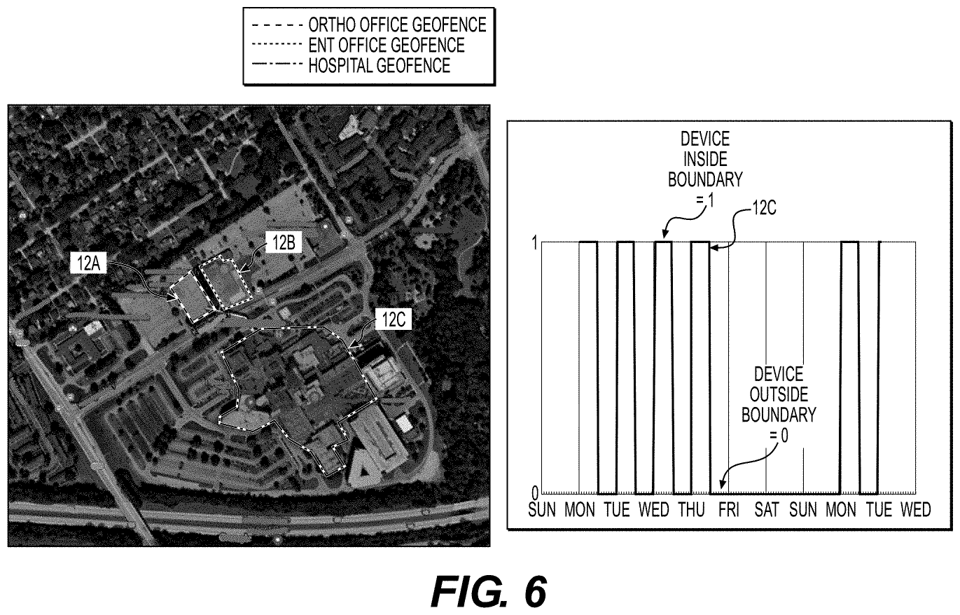

In some embodiments, if there is an above average readmissions in a certain department or facility, the recommendation 328 may include conducting an internal or external quality review. In addition, if there is a large amount of patients leaving a specific department or facility associated with one medical provider and going to a specific department or facility associated with another medical provider, the recommendation 328 may also may include conducting an internal or external quality review. Frequency readings may be visually illustrated as a diagram on a monitor in a Fourier transform or wavelet transform (see FIGS. 6 through 12).

In accordance with another aspect of the disclosure, the recommendation 328 may further include facilitating coordinated responses between more than one medical campus, building, and/or department that may be under the same or different ownership. For example, in large scale emergency situations, such as a pandemic, it may be beneficial to ensure that all resources are fully utilized and that one medical campus, building, or department is not receiving excess patients while another medical campus, building, or department is underutilized. As such, the recommendation may include where and when to send patients and where and when not to send patients, for example, based on employee to patient ratio, room availability, or other resource availability. In some embodiments, health care resources associated with a virtual boundary is further saved in the memory or the server.

In some embodiments, in instances where one type of medical condition is contagious and can greatly impact other patients with different medical conditions, the coordinated response recommendation may further provide instructions on localizing patients with the contagious medical condition to a designated medical campus, building, or department. The localizing of patients recommendation may further include associating a virtual boundary 12 with the medical campus, building, or department that has been designated to treat that specific type of contagious medical condition. A movement timeline of and identifier categorized as a patient between virtual boundaries 12 before or after entering the associated virtual boundary 12 may also be provided such that the generated recommendation can further include administering tests to individuals who were in the same virtual boundaries 12 at the same time and may have come into contact with a patient or other person carrying a contagious medical condition. In addition to providing recommendations on patient movement, movement of other personnel may be recommended. For example, if an employee or other individual is working or visiting within a virtual boundary 12 associated with the contagious medical condition, the generated recommendation may further include administering tests on the employee or other personnel and coordinating/isolating parking areas and travel paths to the associated virtual boundary 12 so that there is no overlap between persons entering the associated virtual boundary 12 and other personnel.

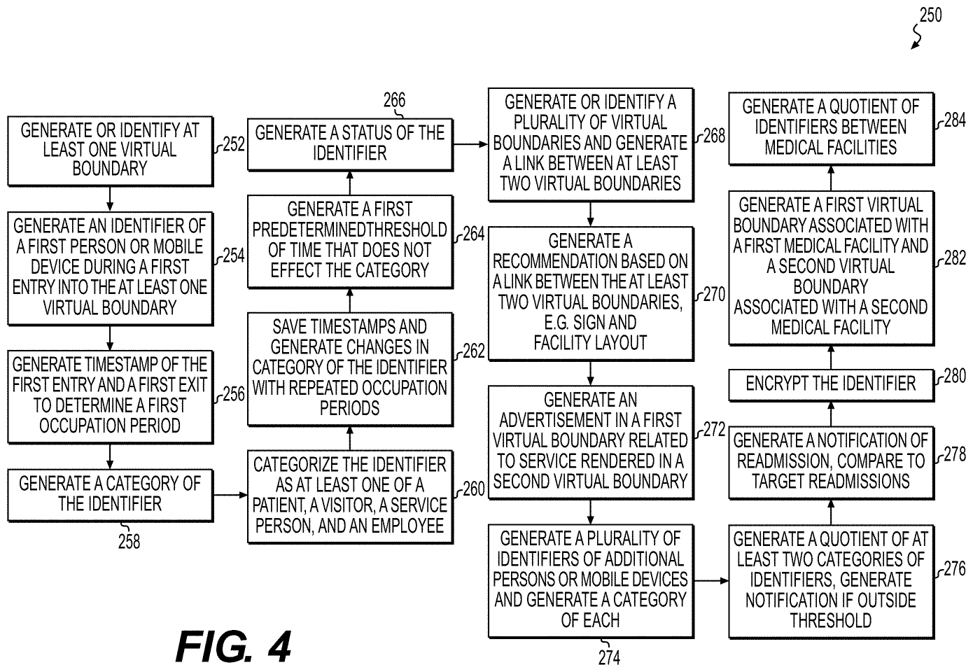

FIG. 5B may be a continuation of FIG. 5A and provides an example flow-chart illustrating additional method steps 350 that may be used with or alternatively to categorizing steps 316 or 318, include providing a series of timing thresholds and passage of time data 317 to determine the category of a person or mobile device. In some embodiments, FIG. 5B may be a continuation of FIG. 4 and may be used with or alternatively to categorizing steps steps 258 through 266 and 274. Moreover, the categorizing steps may incorporate data provided by the server as described herein. In some embodiments, the steps provided in FIG. 5B may be carried about by the processor 220 and/or one of the computing devices.

In some embodiments, the recommendations provided herein may be generated on a mobile device, a computing device, or a screen located within the virtual boundary publically directed to occupants therein. The recommendations provided herein may be generated as a visual notification or an auditory notification. The recommendation and categorization steps described in accordance with one of the methods may be implemented in any of the methods described herein. As such, unless contradictory, steps of one method may be interchanged between methods.

In some embodiments, the system 10, circuit 200, and/or the controller 218 may perform the methods described herein. However, the methods described herein as performed by the system 10, circuit 200, and/or the controller 218 are not meant to be limiting, and any type of software executed on a controller or processor can perform the methods described herein without departing from the scope of this disclosure. For example, a controller, such as a processor executing software within a computing device, can perform the methods described herein.

FIG. 6 provides an example diagram that illustrates a series of timestamped entries and exits of a person or mobile device 18 within three separate virtual boundaries 12A through 12C, including a first virtual boundary 12A surrounding an orthopedic department, a second virtual boundary 12B surrounding an ENT department, and a third virtual boundary 12C surrounding a hospital building. The example diagram may be illustrated as shown on a user interface located on one or both of the local computing device 14, the remote computing device 16, the mobile device 18, or combinations thereof. The diagram may be generated by the processor 220 and/or one of the computing devices.

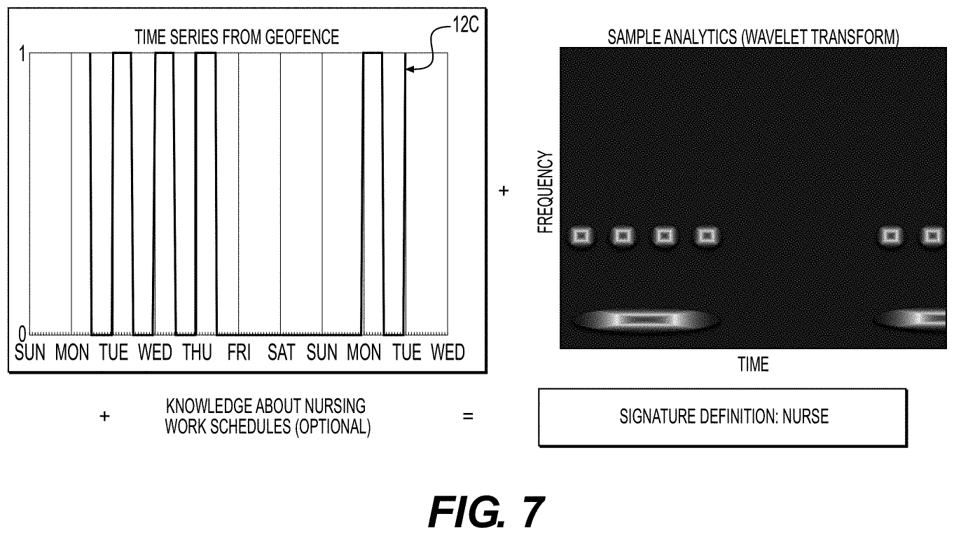

FIG. 7 is a continuation of FIG. 6 and provides a subpopulation diagram based on a series of timestamped entries and exits of a person or mobile device 18 within at least one of the three separate virtual boundaries 12A through 12C. The example diagram may be illustrated as shown on a user interface located on one or both of the local computing device 14, the remote computing device 16, the mobile device 18, or combinations thereof. The diagram may be generated by the processor 220 and/or one of the computing devices.

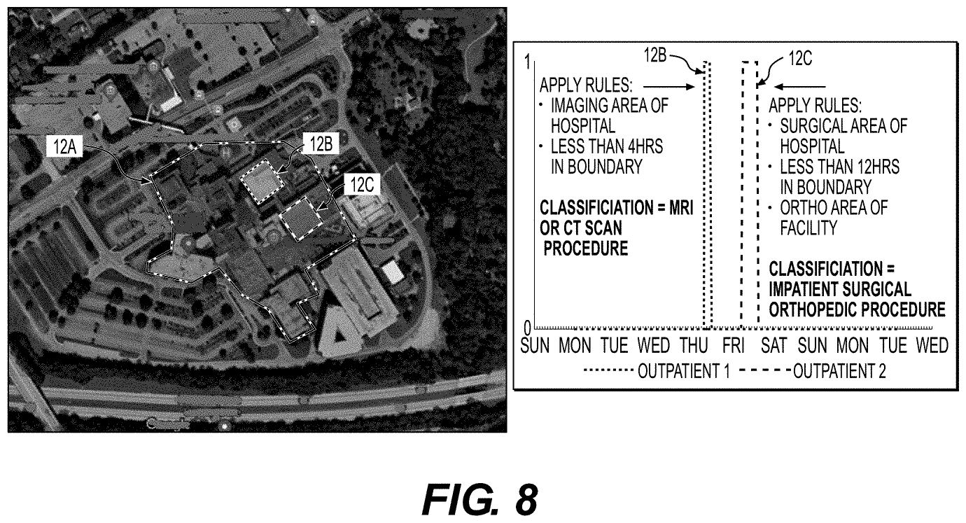

FIG. 8 provides another example diagram that illustrates a series of timestamped entries and exits of a person or mobile device 18 within three separate virtual boundaries 12A through 12C, including a first virtual boundary 12A surrounding an entire medical campus, a second virtual boundary 12B surrounding an MRI department (see operations schedule data 236) and located within 12A, and a third geofence 12C surrounding a surgical department and located within 12A. Both re-entry into the medical campus and the specific departments can be monitored for occupation periods and frequency of re-entry. In addition, the types of treatment received may be extrapolated. The example diagram may be illustrated as shown on a user interface located on one or both of the local computing device 14, the remote computing device 16, the mobile device 18, or combinations thereof. The diagram may be generated by the processor 220 and/or one of the computing devices.

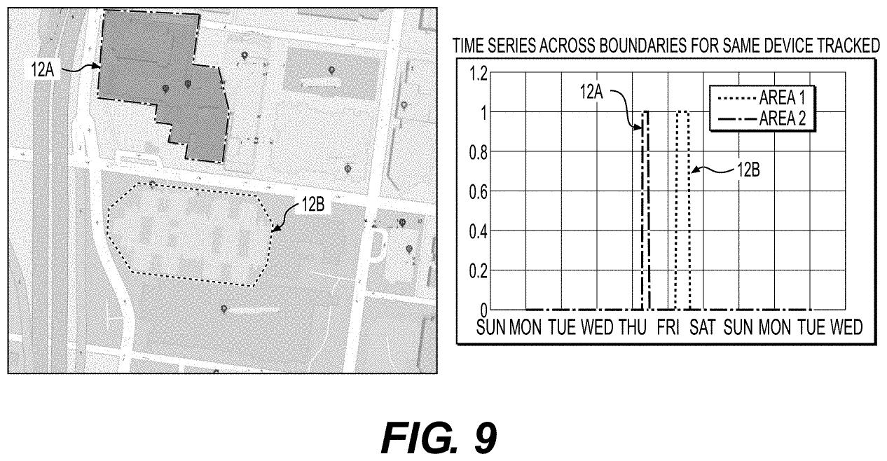

FIG. 9 provides another example diagram that illustrates a series of timestamped entries and exits of a person or mobile device 18 within two non-overlapping separate virtual boundaries 12A and 12B, including a first virtual boundary 12A surrounding first department and a second virtual boundary 12B surrounding second department. Entry into both departments can be monitored for the one person or mobile device that crosses multiple virtual boundaries. The example diagram may be illustrated as shown on a user interface located on one or both of the local computing device 14, the remote computing device 16, the mobile device 18, or combinations thereof. The diagram may be generated by the processor 220 and/or one of the computing devices.

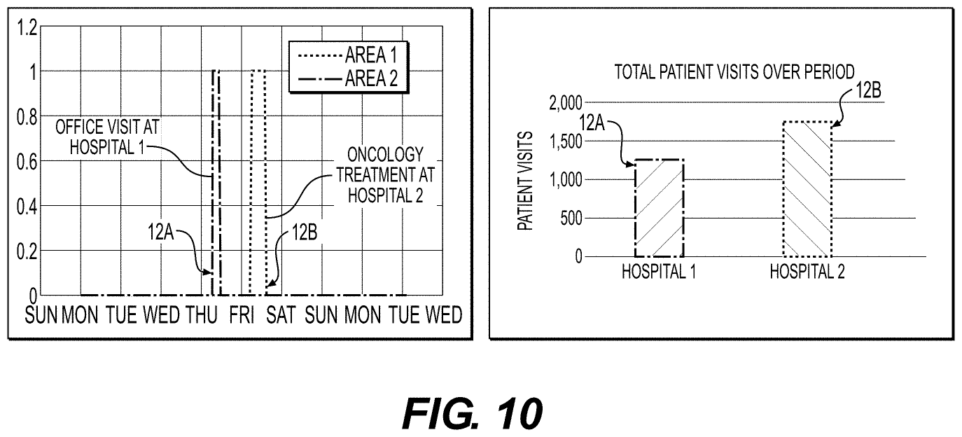

FIG. 10 provides another example diagram that illustrates a series of timestamped entries and exits of a person or mobile device 18 within two non-overlapping separate virtual boundaries 12A and 12B, including a first virtual boundary 12A surrounding first department and a second virtual boundary 12B surrounding second department owned by a different organization. Entry into both departments can be monitored (see proximity data 334 and meta data 340) for one person or mobile device that crosses virtual boundaries associated with different organizations or service providers. As such, the total number of virtual boundary crossings can be monitored and compiled as historical data 320 and meta data 322. The example diagram may be illustrated as shown on a user interface located on one or both of the local computing device 14, the remote computing device 16, the mobile device 18, or combinations thereof. The diagram may be generated by the processor 220 and/or one of the computing devices.

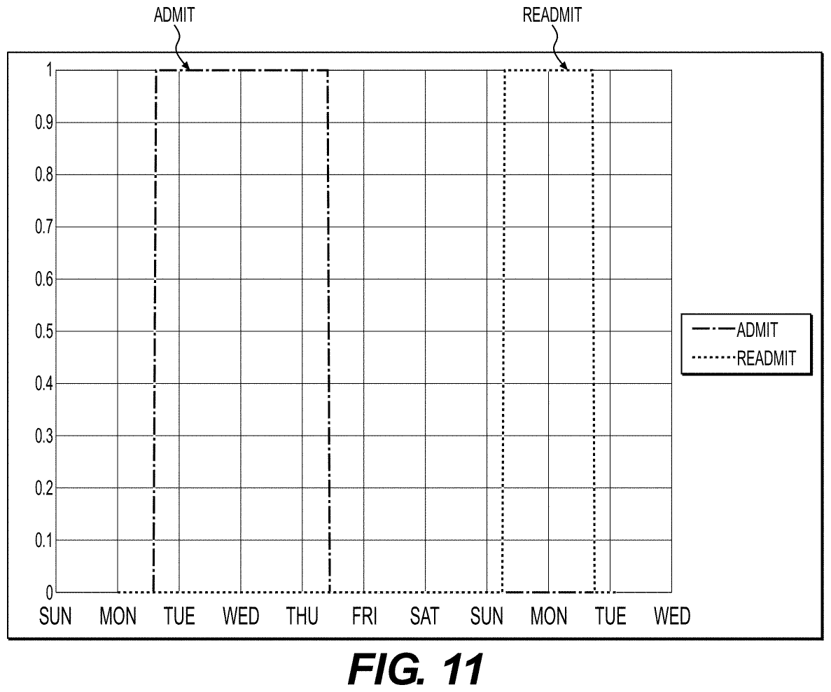

FIG. 11 provides another example diagram that illustrates a series of timestamped entries and exits of a person or mobile device 18 within the same virtual boundary 12A, the timestamps being separated by categorized initial admission and readmissions (see historical data 342). The example diagram may be illustrated as shown on a user interface located on one or both of the local computing device 14, the remote computing device 16, the mobile device 18, or combinations thereof. The diagram may be generated by the processor 220 and/or one of the computing devices.

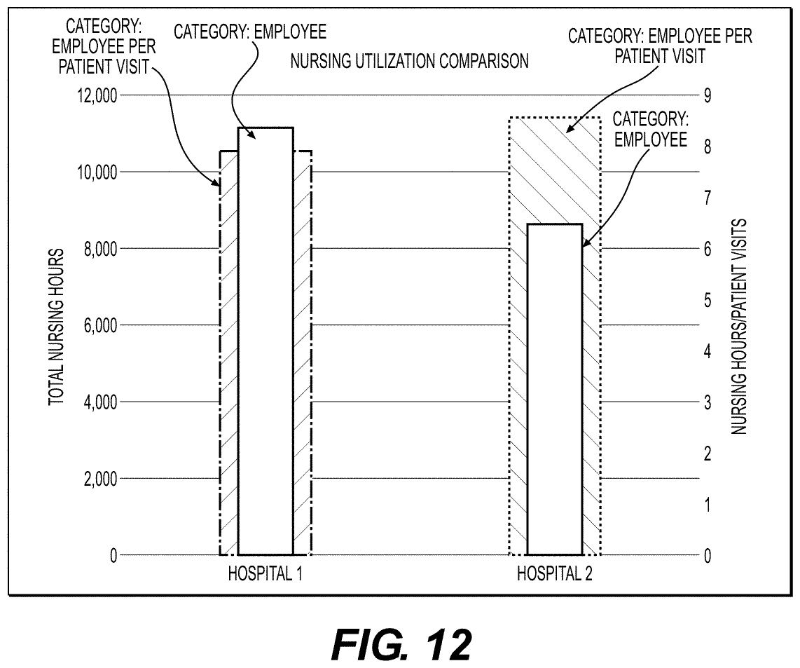

FIG. 12 provides another example diagram that illustrates a series of timestamped entries and exits of a first category of persons or mobile device 18 (identifiers) and a second category of person or mobile devices 18 (identifiers) within two non-overlapping virtual boundaries 12A, 12B, the categories being separated by patients and employees (see meta data 340 and historical data 242). As such, recommendations can be made based on relative staffing levels, e.g., to relocate staff in a location that is overstaffed or increase staff in a location that is understaffed. The example diagram may be illustrated as shown on a user interface located on one or both of the local computing device 14, the remote computing device 16, the mobile device 18, or combinations thereof. The diagram may be generated by the processor 220 and/or one of the computing devices.

It should be appreciated that other beacon or location-aware technologies may be used without departure from the subject disclosure. These additional location-aware technologies may include sensors and methods for calculating the geographical position of a person or object. While the terms geofence and geoframe have been used in relation to example embodiments, these embodiments may also use other technologies, such as, GPS, assisted GPS (A-GPS), Wi-Fi, Enhanced Observed Time Difference (E-OTD), Enhanced GPS (E-GPS) and other technologies. Unless otherwise explicitly limited, the term “location-aware” or “virtual” should be understood to include any technology that would signal geographical positions of persons or objects.

It should be appreciated that the foregoing description of the embodiments has been provided for purposes of illustration. In other words, the subject disclosure it is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varies in many ways. Such variations are not to be regarded as a departure from the disclosure, and all such modifications are intended to be included within the scope of disclosure.

What is claimed is:

1. A monitoring system for at least one healthcare facility comprising:
a memory; and
a processor, wherein the memory includes instructions executable by the processor to:
generate or identify at least one virtual boundary around at least a portion of the at least one healthcare facility;
generate an identifier of a person or a mobile device during a first entry into the at least one virtual boundary;
generate timestamp of the first entry and a timestamp of the first exit of the identifier to determine a first occupation period;
generate a category of the identifier based on at least one of the occupation period, a frequency of reentries into the at least one virtual boundary, or a time of the day associated with at least one of the timestamps.

2. The system of claim 1, wherein the category of the identifier includes at least one of a patient, a visitor, a service person, and an employee.

3. The system of claim 2, wherein the frequency of reentries into the at least one virtual boundary includes additional occupation periods.

4. The system of claim 3, wherein the timestamps and occupation periods associated with the identifier are saved in the memory and the processor is further caused to generate changes in the category of the identifier associated with the continuing timestamps and occupation periods after then first entry and the first exit.

5. The system of claim 4, wherein if an occupation period or a period of time between occupation periods is less than a first predetermined threshold it is not used to generate a category or a change in category.

6. The system of claim 5, wherein if the occupation period or the period of time between occupation periods is less than the first predetermined threshold the processor is further caused to generated a status of the identifier including at least one of the identifier being lost in the at least one medical facility, the identifier leaving for food, the identifier looking for parking, or the identifier traversing the at least one virtual boundary to enter an area outside of the at least one virtual boundary.

7. The system of claim 6, wherein the at least one virtual boundary includes a plurality of virtual boundaries and the processor is further caused to generate a link between the plurality of virtual boundaries every time an identifier travels between two or more of the virtual boundaries.

8. The system of claim 7, wherein the processor generates a recommendation based on the link between at least two virtual boundaries for at least one of facility layout or the placement of signs to guide traversal between the at least two virtual boundaries.

9. The system of claim 8, wherein the recommendation for the placement of signs includes a recommendation in or near a first virtual boundary of the at least two virtual boundaries related to services rendered in a second virtual boundary of the at least two virtual boundaries and the recommendation includes signs for identifiers traveling to the second virtual boundary to avoid the first virtual boundary.

10. The system of claim 9, wherein the sign is generated digitally on a screen in the medical facility or on the mobile device associated with the identifier.

11. The system of claim 7, wherein the processor generates an advertisement in a first virtual boundary of the at least two virtual boundaries related to services rendered in a second virtual boundary of the at least two virtual boundaries.

12. The system of claim 11, wherein the advertisement is generated digitally in response to a time of day associated with the travel between the at least two virtual boundaries or a time stamp of entry of the identifier into one of the plurality of virtual boundaries.

13. The system of claim 4, wherein the processor generates a plurality of additional identifiers of additional persons or a mobile devices and further generates a category of each of the identifiers based on at least one of the occupation period, a frequency of reentries into the virtual boundary, or a time of the day associated with at least one of the timestamps.

14. The system of claim 13, wherein the processor generates a quotient of at least two categories of identifiers and further generates a recommendation for a target quotient of the at least two categories of identifiers.

15. The system of claim 14, wherein the processor generates a notification if a quotient between the at least two categories of identifiers is outside of a predetermined threshold.

16. The system of claim 4, wherein the processor is further caused to generate a notification of readmission when a predetermined length of time passes between occupation periods.

17. The system of claim 16, wherein the processor is further caused to generate a recommendation for a target limit of readmissions.

18. The system of claim 1, wherein the processor is further caused to encrypt the identifier so that the identifier does not include any personal information that could be used to ascertain a personal identity.

19. The system of claim 1, wherein the at least one virtual boundary includes a first virtual boundary around at least a portion of a first healthcare facility and a second virtual boundary around at least a portion of a second healthcare facility and the processor generates a plurality of additional identifiers of additional persons or a mobile devices and further generates a category of each of the identifiers based on at least one of the occupation period, a frequency of reentries into the virtual boundary, or a time of the day associated with at least one of the timestamps.

20. The system of claim 19, wherein the processor is further caused to generate a quotient between the number of identifiers associated with the first medical facility and the number of identifiers associated with the second medical facility.
